SEQUENCE LISTING (24 sequences)

SEQ ID NO: 1 - DNA, 954 bp, Homo sapien
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgcgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 2 - DNA, 954 bp, Homo sapien
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 3 - protein, 317 aa, Homo sapien [sequence omitted]

SEQ ID NO: 4 - DNA, 1144 bp, Homo sapien
agagacgacccgacccgctagaagactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgca

SEQ ID NO: 5 - protein, 317 aa, Homo sapien [sequence omitted]

SEQ ID NO: 6 - protein, 317 aa, Artificial sequence
  Synthetic
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVSLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 7 - DNA, 954 bp, Artificial sequence
  Synthetic
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgagcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 8 - protein, 317 aa, Artificial sequence
  Synthetic
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVSLASHLRKLRKRLLRDADDLQKCLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 9 - DNA, 954 bp, Artificial sequence
  Synthetic
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgagcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 10 - DNA, 954 bp, Artificial sequence
  Synthetic
atgaaggtgctgtgggccgccctgctggtgaccttcctggccggctgccaggccaaagtcgaacaggccgtcgagaccgagcccgagcccgagctgcgccagcagaccgagtggcagagcggccagcgctgggagctggccctgggccgcttctgggactacctgcgctgggtgcagaccctgagcgagcaggtgcaggaggagctgctgagcagccaggtgacccaggagctgcgcgccctgatggacgagaccatgaaagaactcaaagcttataagagcgagctggaggagcagctgacccccgtggccgaggagacccgcgcccgcctgagcaaggagctgcaggccgcccaggcccgcctgggcgccgacatggaggacgtgtgcggccgcctggtgcagtaccgcggcgaggtgcaggccatgctgggccagagcaccgaggagctgcgcgtgagcctggccagccacctgcgcaagctgcgcaagcgcctgctgcgcgacgccgacgacctgcagaagcgcctggccgtgtaccaggccggcgcccgcgagggcgccgagcgcggcctgagcgccatccgcgagcgcctgggccccctggtggagcagggccgcgtgcgcgccgccaccgtgggcagcctggccggccagcccctgcaggagcgcgcccaggcctggggcgagcgcctgcgcgcccgcatggaggagatgggcagccgcacccgcgaccgcctggacgaggtgaaggagcaggtggccgaggtgcgcgccaagctggaggagcaggcccagcagatccgcctgcaggccgaggccttccaggcccgcctgaagagctggttcgagcccctggtggaggacatgcagcgccagtgggccggcctggtggagaaggtgcaggccgccgtgggcaccagcgccgcccccgtgcccagcgacaaccactaa

SEQ ID NO: 11 - DNA, 954 bp, Artificial sequence
  Synthetic
atgaaggtgctgtgggccgccctgctggtgaccttcctggccggctgccaggccaaagtcgaacaggccgtcgagaccgagcccgagcccgagctgcgccagcagaccgagtggcagagcggccagcgctgggagctggccctgggccgcttctgggactacctgcgctgggtgcagaccctgagcgagcaggtgcaggaggagctgctgagcagccaggtgacccaggagctgcgcgccctgatggacgagaccatgaaagaactcaaagcttataagagcgagctggaggagcagctgacccccgtggccgaggagacccgcgcccgcctgagcaaggagctgcaggccgcccaggcccgcctgggcgccgacatggaggacgtgtgcggccgcctggtgcagtaccgcggcgaggtgcaggccatgctgggccagagcaccgaggagctgcgcgtgagcctggccagccacctgcgcaagctgcgcaagcgcctgctgcgcgacgccgacgacctgcagaagtgcctggccgtgtaccaggccggcgcccgcgagggcgccgagcgcggcctgagcgccatccgcgagcgcctgggccccctggtggagcagggccgcgtgcgcgccgccaccgtgggcagcctggccggccagcccctgcaggagcgcgcccaggcctggggcgagcgcctgcgcgcccgcatggaggagatgggcagccgcacccgcgaccgcctggacgaggtgaaggagcaggtggccgaggtgcgcgccaagctggaggagcaggcccagcagatccgcctgcaggccgaggccttccaggcccgcctgaagagctggttcgagcccctggtggaggacatgcagcgccagtgggccggcctggtggagaaggtgcaggccgccgtgggcaccagcgccgcccccgtgcccagcgacaaccactaa

SEQ ID NO: 12 - DNA, 21 bp, Artificial sequence
  Synthetic
ttgtaggccttcaactccttc

SEQ ID NO: 13 - DNA, 21 bp, Artificial sequence
  Synthetic
gaaggagttgaaggcctacaa

SEQ ID NO: 14 - DNA, 58 bp, Artificial sequence
  Synthetic
ttgtaggccttcaactccttccatctgtggcttcactgaaggagttgaaggcctacaa

SEQ ID NO: 15 - DNA, 152 bp, Artificial sequence
  Synthetic
ttgtcatcctcccacggtggccatttgttccatgtgagtgctagtaacaggccttgtgtcctttgtaggccttcaactccttccatctgtggcttcactgaaggagttgaaggcctacaagacaacagcatacagccttcagcaagcctcca

SEQ ID NO: 16 - DNA, 21 bp, Artificial sequence
  Synthetic
ctccaccgcttgctccacctt

SEQ ID NO: 17 - DNA, 21 bp, Artificial sequence
  Synthetic
aaggtggagcaagcggtggag

SEQ ID NO: 18 - DNA, 58 bp, Artificial sequence
  Synthetic
ctccaccgcttgctccaccttagtgaagccacagatgaaggtggagcaagcggtggag

SEQ ID NO: 19 - DNA, 152 bp, Artificial sequence
  Synthetic
tggaggcttgctgaaggctgtatgctgttgtcctccaccgcttgctccaccttagtgaagccacagatgaaggtggagcaagcggtggagaggacacaaggcctgttactagcactcacatggaacaaatggccaccgtgggaggatgacaa

SEQ ID NO: 20 - DNA, 19 bp, Artificial sequence
  Synthetic
tttgtaggccttcaactcc

SEQ ID NO: 21 - DNA, 19 bp, Artificial sequence
  Synthetic
ggagttgaaggcctacaaa

SEQ ID NO: 22 - DNA, 54 bp, Artificial sequence
  Synthetic
tttgtaggccttcaactccagtgaagccacagatgggagttgaaggcctacaaa

SEQ ID NO: 23 - DNA, 148 bp, Artificial sequence
  Synthetic
tggaggcttgctgaaggctgtatgctgttgtctttgtaggccttcaactccagtgaagccacagatgggagttgaaggcctacaaaaggacacaaggcctgttactagcactcacatggaacaaatggccaccgtgggaggatgacaa

SEQ ID NO: 24 - DNA, 145 bp, Unknown
  AAV2
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

(19) United States
(12) Patent Application Publication
(10) Pub. No.: US 2023/0405149 A1
(43) Pub. Date: Dec. 21, 2023

(54) GENE THERAPIES FOR NEURODEGENERATIVE DISEASE

(71) Applicant: Prevail Therapeutics, Inc., New York, NY (US)

(72) Inventors: Asa Abeliovich, NEW YORK, NY (US); Sitharthan Kamalakaran, New York, NY (US); Benjamin Shykind, New York, NY (US); Edmund c. Schwartz, New York, NY (US); Anindya Kumar Sen, Revere, MA (US)

(73) Assignee: Prevail Therapeutics, Inc., New York, NY (US)

(21) Appl. No.: 18/038,247

(22) PCT Filed: Nov. 24, 2021
(86) PCT No.: PCT/US2021/060731

§ 371 Date: May 23, 2023

Related U.S. Application Data
(60) Provisional application No. 63/118,060, filed on Nov. 25, 2020.

(51) Int. Cl. A61K 48/00; A61P 25/28

(52) U.S. Cl. CPC A61K 48/005; A61P 25/28; A61K 48/0008; A61K 48/0075

(57) ABSTRACT

The disclosure relates, in some aspects, to compositions and methods for treatment of neurodegenerative disease, for example Alzheimer's disease. In some embodiments, the disclosure provides expression constructs comprising a transgene encoding an APOE Christchurch (e.g., APOE3ch and/or APOE2ch) protein isoform or a portion thereof, an inhibitory nucleic acid targeting an APOE gene or a portion thereof, or any combination of the foregoing. In some embodiments, the disclosure provides methods of treating Alzheimer's disease by administering an expression construct to a subject in need thereof.

### RELATED APPLICATION

This Application is a national stage filing under 35 U.S.C. § 371 of international PCT application PCT/US2021/060731, filed Nov. 24, 2021, which claims priority under 35 U.S.C. § 119(e) to U.S. provisional patent application, U.S. Ser. No. 63/118,060, filed Nov. 25, 2020, the entire contents of each of which are incorporated by reference herein.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on May 17, 2023, is named P109470016US01-SEQ-KZM and is 24,131 bytes in size.

### BACKGROUND

Alzheimer's disease (AD) is the most common form of dementia, affecting more than 5 million people in the United States alone. Alzheimer's disease is an irreversible, progressive brain disorder characterized by the presence of abnormal protein deposits throughout the brain, which inhibit neuronal function, disrupt connections between neurons, and ultimately result in cell death. These deposits comprise plaques of amyloid-β and tangles formed by phosphorylated-tau proteins. Patients with mild AD experience memory loss, leading to wandering, difficulty handling money, repeating questions, and personality and behavior changes. Moderate AD patients exhibit increased memory loss, leading to confusion and difficulty recognizing friends and family, inability to learn new things, hallucinations, delusions, and paranoia. Patients with severe AD cannot communicate and are completely depending on others for their care. Ultimately, protein plaques and tangles spread throughout the brain, leading to significant tissue shrinkage.

### SUMMARY

Most Alzheimer's disease (AD) patients have late-onset AD, in which symptoms being to appear in the subject's mid-60's. The apolipoprotein E (APOE) gene is involved in the development of late-onset AD. APOE has several isoforms, including APOE2, which is protective against AD, and APOE4, which is associated with increased risk for developing late-onset AD. Homozygous patients who carry two copies of APOE4 (e.g., subjects that are APOE4^{+/+}) are at an even greater risk of developing late-onset AD as compared to heterozygous patients who carry one copy of APOE4 and one copy of either APOE2 or APOE3. In addition, Presenilin 1 (PSEN1) mutation (e.g., PSEN1 E280A mutation) is associated with autosomal-dominant AD. It was found that a PSEN1 E280A mutation carrier who was homozygous for the APOE3 Christchurch mutation (e.g., APOE3 R136S mutation) developed cognitive impairment much later than PSEN1 E280A mutation carriers who are not homozygotes of APOE3 Christchurch mutation (e.g., APOE3 R136S mutation).

Aspects of the disclosure relate to compositions and methods for treating a subject having or suspected of having AD (e.g., ADAD). The disclosure is based, in part, on expression constructs encoding an APOE Christchurch protein (e.g., APOE3ch protein and/or APOE2ch protein). In some aspects, the expression construct also encodes an inhibitory RNA (e.g., shRNA, miRNA, amiRNA, etc.) that targets an AD-associated gene (e.g., APOE, such as APOE4, APOE3, and/or APOE2).

In some aspects, the present disclosure provides an isolated nucleic acid comprising an expression construct comprising a nucleic acid sequence encoding an APOE Christchurch protein.

In some embodiments, the APOE Christchurch protein is an APOE2 Christchurch protein. In some embodiments, the APOE2 Christchurch protein comprises an amino acid sequence at least 80% identical to SEQ ID NO: 8. In some embodiments, the expression construct encoding the APOE2 Christchurch protein comprises a nucleic acid sequence at least 80% identical to SEQ ID NO: 9. In some embodiments, the APOE Christchurch protein is an APOE3 Christchurch protein. In some embodiments, the APOE3 Christchurch protein comprises an amino acid sequence at least 80% identical to an amino acid sequence of SEQ ID NO: 6. In some embodiments, the expression construct encoding the APOE3 Christchurch protein comprises a nucleic acid sequence at least 80% identical to SEQ ID NO: 7.

In some embodiments, the expression construct further comprises a nucleic acid sequence encoding an inhibitory nucleic acid that inhibits expression or activity of one or more APOE gene isoforms (e.g., APOE4, APOE3, APOE2, etc.). In some embodiments, the expression construct further comprises a nucleic acid sequence encoding an inhibitory nucleic acid that inhibits expression or activity of APOE4. In some embodiments, the expression construct further comprises a nucleic acid sequence encoding an inhibitory nucleic acid that inhibits expression or activity of APOE2. In some embodiments, the expression construct further comprises a nucleic acid sequence encoding an inhibitory nucleic acid that inhibits expression or activity of APOE3. In some embodiments, the expression construct further comprises a nucleic acid sequence encoding an inhibitory nucleic acid that inhibits expression or activity of APOE4 and APOE2. In some embodiments, the expression construct further comprises a nucleic acid sequence encoding an inhibitory nucleic acid that inhibits expression or activity of APOE4, APOE3, and APOE2. In some embodiments, the inhibitory nucleic acid is encoded by a sequence set forth in any one of SEQ ID NOs: 12-23.

In some embodiments, the expression construct further comprises a first promoter operably linked to the nucleic acid sequence encoding the APOE Christchurch protein. In some embodiments, the first promoter is operably linked to the nucleic acid sequence encoding an inhibitory nucleic acid that inhibits expression or activity of one or more APOE isoforms (e.g., APOE4, APOE3, APOE2, etc.). In some embodiments, the expression construct further comprises a second promoter operably linked to the nucleic acid sequence encoding an inhibitory nucleic acid that inhibits expression or activity of one or more APOE isoforms (e.g., APOE4, APOE3, APOE2, etc.). In some embodiments, the first promoter and/or the second promoter is independently a chicken-beta actin (CBA) promoter, a CAG promoter, a CD68 promoter, or a JeT promoter.

In some embodiments, the expression construct is flanked by adeno-associated virus (AAV) inverted terminal repeats (ITRs). In some embodiments, the ITRs are AAV2 ITRs.

In some embodiments, the isolated nucleic acid comprises the sequence set forth in any one of SEQ ID NOs: 6-11.

In some aspects, the present disclosure provides a vector comprising the isolated nucleic acid described herein. In some embodiments, the vector is a plasmid. In some embodiments, the vector is a viral vector. In some embodiments, the viral vector is a recombinant AAV (rAAV) vector or a Baculovirus vector.

In some aspects, the present disclosure provides a recombinant adeno-associated virus (rAAV) comprising: (i) an AAV capsid protein; and (ii) an isolated nucleic acid or the vector as described herein.

In some embodiments, the AAV capsid protein is capable of crossing the blood-brain barrier. In some embodiments, the AAV capsid protein is an AAV9 capsid protein or an AAVrh.10 capsid protein. In some embodiments, the rAAV transduces neuronal cells and non-neuronal cells of the central nervous system (CNS).

In some aspects, the present disclosure provides a host cell comprising the isolated nucleic acid, the vector, or the rAAV as described herein.

In some aspects, the present disclosure provides a composition comprising the isolated nucleic acid, the vector, or the rAAV as described herein.

In some embodiments, the composition is a pharmaceutical composition further comprises a pharmaceutically acceptable carrier.

In some aspects, the present disclosure provides a method comprising administering to a subject having or suspected of having Alzheimer's disease an isolated nucleic acid, the vector, the rAAV, or the composition as described herein.

In some embodiments, the administration comprises direct injection to the CNS of the subject. In some embodiments, the direct injection comprises intracerebral injection, intraparenchymal injection, intrathecal injection, or any combination thereof. In some embodiments, the direct injection to the CNS of the subject comprises convection enhanced delivery (CED). In some embodiments, the administration comprises peripheral injection. In some embodiments, the peripheral injection comprises intravenous injection.

In some embodiments, the subject has or is suspected of having autosomal dominant Alzheimer's disease (ADAD). In some embodiments, the subject has at least one mutation in the PSEN1 gene. In some embodiments, the mutation in the PSEN1 gene causes an E280A mutation in the presenilin 1 protein. In some embodiments, the subject is not a homozygote for APOE3 Christchurch mutation, wherein the APOE3 Christchurch mutation causes a R136S mutation in APOE3 protein. In some embodiments, the administration results in delayed onset of mild cognitive impairment (MIC) compared to subjects not receiving the administration.

### BRIEF DESCRIPTION OF THE DRAWINGS

FIG. 1 shows a schematic depicting one embodiment of a vector encoding an APOE Christchurch variant protein.

FIG. 2 shows a multiple sequence alignment of wild-type APOE2, APOE2_Christchurch, and APOE3_Christchurch. SEQ ID NOS: 3, 8, and 6 are shown, top to bottom.

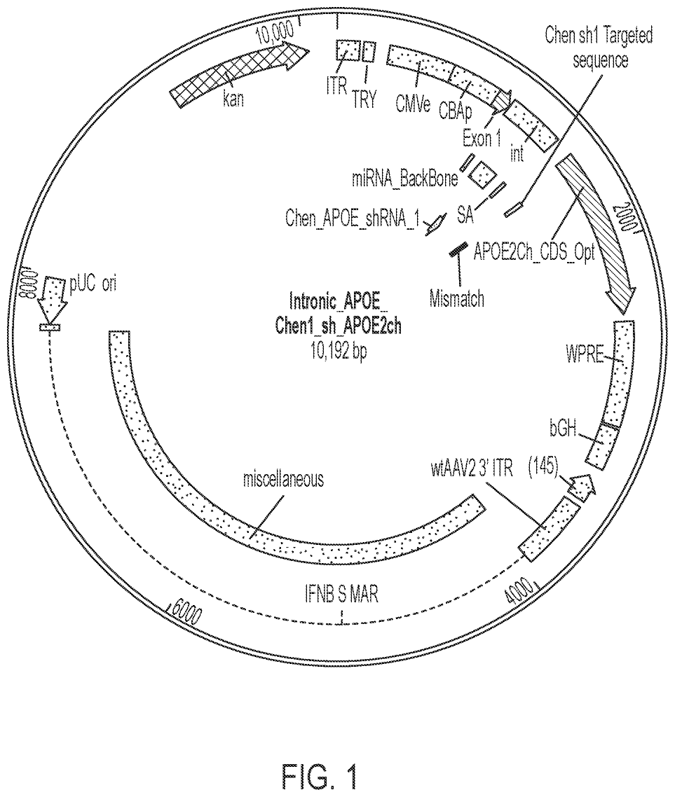

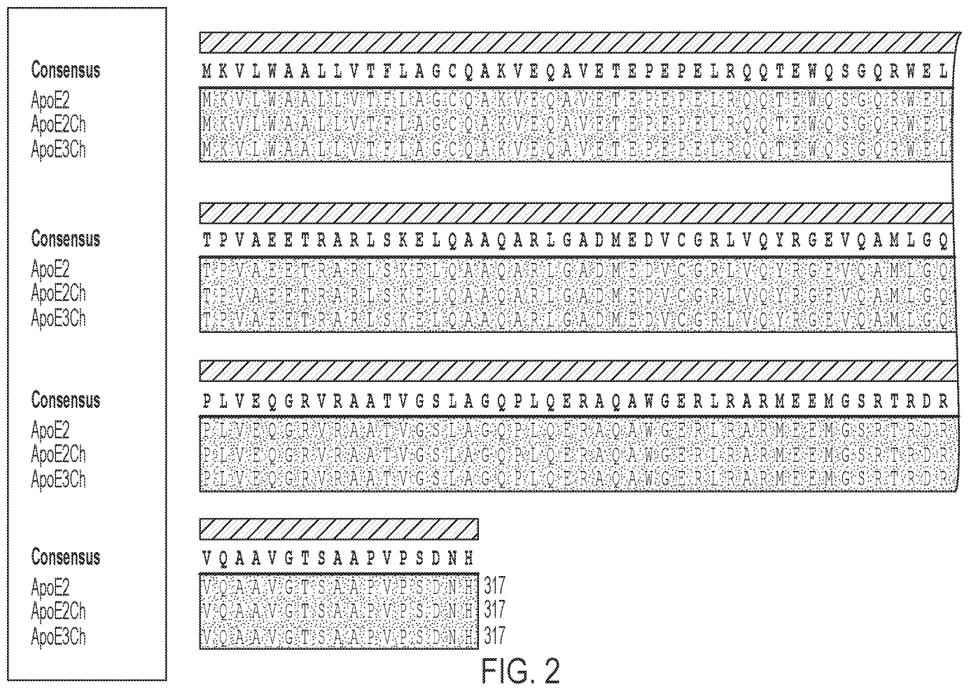

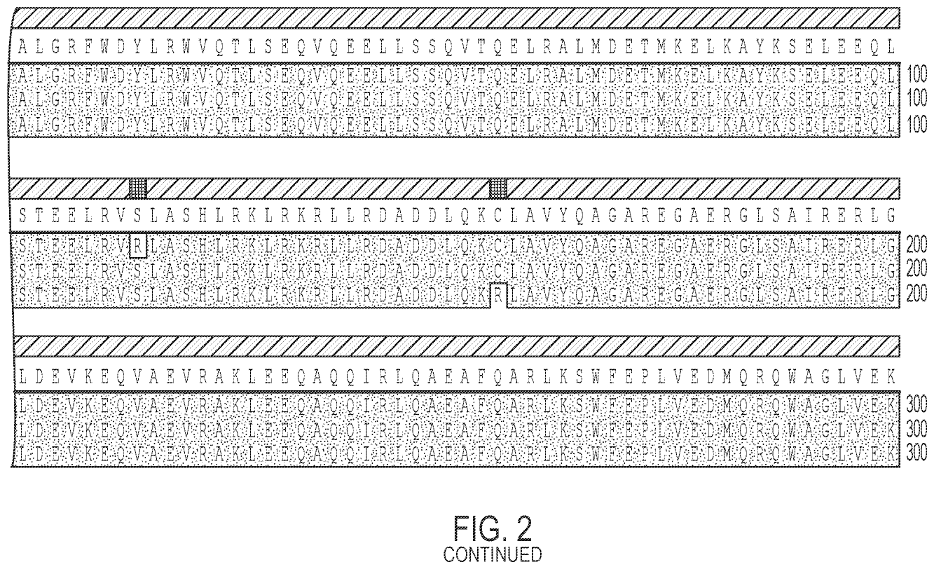

### DETAILED DESCRIPTION

The disclosure is based, in part, on compositions and methods for expression of combinations of AD-associated gene products in a subject. A gene product can be a protein, a fragment (e.g., portion) of a protein, an interfering nucleic acid that inhibits an AD-associated gene, etc. In some embodiments, a gene product is a protein or a protein fragment encoded by an AD-associated gene. In some embodiments, a gene product is an inhibitory nucleic acid (e.g., shRNA, siRNA, miRNA, amiRNA, etc.) that inhibits an AD-associated gene.

An AD-associated gene refers to a gene encoding a gene product that is genetically, biochemically or functionally associated with Alzheimer's disease (AD). For example, individuals having at least one copy of Presenilin 1 (PSEN1) comprising an E280A mutation are at an increased risk of develop autosomal-dominant Alzheimer's disease (ADAD). In some embodiments, APOE3 Christchurch mutation homozygosity (APOE3ch^{+/+}) exhibits a neuroprotective effect in ADAD patients with the Presenilin 1 (PSEN1) E280A mutation. In other instances, individuals having at least one copy of APOE4 are at an increased risk of developing late-onset AD. In another example, APOE2 exhibits a neuroprotective effect in mouse models of AD. As used herein, the term “neuroprotective” refers to the preservation of neuronal structure and/or function in a cell or subject relative to the preservation of neuronal structure and/or function in a cell or subject in the absence of neuroprotection (e.g., the absence of a neuroprotective agent or protein).

### Isolated Nucleic Acids and Vectors

An isolated nucleic acid may be DNA or RNA. In some aspects, the disclosure provides an isolated nucleic acid comprising an expression construct comprises a nucleic acid sequence encoding an APOE Christchurch protein (e.g., APOE2 Christchurch protein and/or APOE3 Christchurch protein). Aspects of the disclosure also relate to an isolated nucleic acid comprising an expression construct comprises a nucleic acid sequence encoding an APOE Christchurch protein (e.g., APOE2 Christchurch protein and/or APOE3 Christchurch protein) and nucleic acid sequences encoding one or more inhibitory nucleic acids (e.g., dsRNA, siRNA, miRNA, amiRNA, etc.) that target one or more endogenous APOE gene isoforms (e.g., isoform 2, 3, and/or 4 of the APOE gene).

APOE protein refers to apolipoprotein E, which is a fat binding protein that plays a role in catabolism of triglyceride-rich lipoproteins. There are three major isoforms of APOE, referred to as APOE2, APOE3, and APOE4. Each isoform differs from the others at two positions, amino acid 130 and amino acid 176 (also respectively referred to as positions 112 and 158 when the signal peptide of the protein is excluded). APOE2 contains Cys130/Cys176 and has been observed to be associated with type III hyperlipoproteinemia and other diseases but also plays a neuroprotective role. APOE3 contains Cys130/Arg176 and is the most common APOE allele. APOE4 contains Arg130/Arg176 and has been observed to be associated with late-onset Alzheimer's disease, atherosclerosis, unfavorable outcomes in traumatic brain injury (TBI) and other diseases. In humans, APOE gene is located on chromosome 19. In some embodiments, APOE4 is encoded by a nucleic acid sequence set forth in SEQ ID NO: 1. In some embodiments, APOE2 is encoded by a nucleic acid sequence set forth in SEQ ID NO: 2. In some embodiments, APOE3 is encoded by a nucleic acid sequence set forth in SEQ ID NO: 4.

In some aspects, the present disclosure is based on the surprising discovery that APOE3 Christchurch mutation (e.g., APOE3ch^{+/+}) plays a neuroprotective role in AD patients (e.g., AD patients who are PSEN1 E280A mutation carriers). The APOE Christchurch mutation (APOEch), as described herein, refers to an APOE mutant protein harboring a R136S amino acid substitution associated with mutations in codon 154 of the APOE coding sequence. In some embodiments, the isolated nucleic acid described herein comprises an expression construct encoding an APOE Christchurch protein. In some embodiments, the nucleic acid sequence encoding the APOE Christchurch protein is codon-optimized. In some embodiments, the isolated nucleic acid encodes an APOE3 Christchurch protein, or a fragment thereof. The term “fragment” refers to a portion of a polypeptide or nucleic acid molecule of a reference polypeptide or nucleic acid molecule (e.g., wild type or full-length isoform). In some embodiments, a fragment is a truncation from either end of the reference molecule and has at least 10%, 20%, 30%, 40%, 50%, 60%, 70%, 75%, 80%, 85%, 90%, 95%, 96%, 97%, 98%, 99%, or more sequence identity to the reference molecule (e.g., wild type or full-length isoform). In some embodiments, a fragment contains deletion (e.g., 1, 2, 3, 4, 5, 6, 7, 8, 9, 10, 11, 12, 13, 14, 15, 16, 17, 18, 19, 20, or more deletions) of amino acids or nucleotides across the length of a reference molecule (e.g., wild type or full-length isoform). In some embodiments, the isolated nucleic acid encodes a protein having at least 60%, at least 70%, at least 80%, at least 85%, at least 90%, at least 91%, at least 92%, at least 93%, at least 94%, at least 95%, at least 96%, at least 97%, at least 98%, at least 99%, or 100% identical to amino acid sequence as set forth in SEQ ID NO: 6. In some embodiments, the isolated nucleic acid encodes an APOE2 Christchurch protein, or a fragment thereof. In some embodiments, The isolated nucleic acid encodes a protein having at least 60%, at least 70%, at least 80%, at least 85%, at least 90%, at least 91%, at least 92%, at least 93%, at least 94%, at least 95%, at least 96%, at least 97%, at least 98%, at least 99%, or 100% identical to amino acid sequence as set forth in SEQ ID NO: 8. A protein fragment may comprise about 50%, about 60%, about 70%, about 80% about 90% or about 99% of a protein encoded by an APOEch gene. In some embodiments, a protein fragment comprises between 50% and 99.9% (e.g., any value between 50% and 99.9%) of a protein having the amino acid sequence set forth in SEQ ID NOs: 6 or 8.

In some embodiments, a gene product (e.g., a transgene encoding APOE Christchurch protein) is encoded by a coding portion (e.g., a cDNA) of a naturally occurring gene. In some embodiments, a gene product is a protein (or a fragment thereof) encoded by an APOE gene harboring the APOE Christchurch mutation. In some embodiments, a gene product is a protein (or a fragment thereof) encoded by an APOE3 gene harboring the APOE Christchurch mutation (e.g., APOE3ch). In some embodiments, an APOE3ch gene comprises the nucleic acid sequence at least 60%, at least 70%, at least 80%, at least 85%, at least 90%, at least 91%, at least 92%, at least 93%, at least 94%, at least 95%, at least 96%, at least 97%, at least 98%, at least 99%, or 100% identical to a nucleic acid sequence as set forth in SEQ ID NO: 7. In some embodiments, a gene product is a protein (or a fragment thereof) encoded by an APOE2 gene harboring the APOE Christchurch mutation (e.g., APOE2ch). In some embodiments, an APOE3ch gene comprises the nucleic acid sequence at least 60%, at least 70%, at least 80%, at least 85%, at least 90%, at least 91%, at least 92%, at least 93%, at least 94%, at least 95%, at least 96%, at least 97%, at least 98%, at least 99%, or 100% identical to a nucleic acid sequence as set forth in SEQ ID NO: 9. In some embodiments, the nucleic acid sequence encoding the APOE Christchurch protein is codon optimized. In some embodiments, the codon optimized nucleic acid sequence encoding an APOE3ch protein is at least 60%, at least 70%, at least 80%, at least 85%, at least 90%, at least 91%, at least 92%, at least 93%, at least 94%, at least 95%, at least 96%, at least 97%, at least 98%, at least 99%, or 100% identical to a nucleic acid sequence as set forth in SEQ ID NO: 10. In some embodiments, the codon optimized nucleic acid sequence encoding an APOE2ch protein is at least 60%, at least 70%, at least 80%, at least 85%, at least 90%, at least 91%, at least 92%, at least 93%, at least 94%, at least 95%, at least 96%, at least 97%, at least 98%, at least 99%, or 100% identical to a nucleic acid sequence as set forth in SEQ ID NO: 11.

In some embodiments, an isolated nucleic acid as described herein further comprises a nucleic acid sequence encoding 1, 2, 3, 4, 5, 6, 7, 8, 9, 10, or more inhibitory nucleic acids (e.g., dsRNA, siRNA, shRNA, miRNA, amiRNA, etc.). In some embodiments, an isolated nucleic acid encodes more than 10 inhibitory nucleic acids. In some embodiments, each of the one or more inhibitory nucleic acids targets a different gene or a portion of a gene (e.g., a first miRNA targets a first target sequence of a gene and a second miRNA targets a second target sequence of the gene that is different than the first target sequence). In some embodiments, each of the one or more inhibitory nucleic acids targets the same target sequence of the same gene (e.g., an isolated nucleic acid encodes multiple copies of the same miRNA).

In some embodiments, an isolated nucleic acid encodes a gene product that is an inhibitory nucleic acid that targets (e.g., hybridizes to, or comprises a region of complementarity with) an AD-associated gene (e.g., one or more endogenous APOE gene products, such as one or more APOE4 isoform, APOE3 isoform, and/or APOE2 isoform of the APOE gene). A skilled artisan recognizes that the order of expression of a first gene product (e.g., APOEch protein) and a second gene product (e.g., inhibitory RNA targeting APOE4 isoform of the APOE gene) can generally be reversed (e.g., the inhibitory RNA is the first gene product and APOE2 is the second gene product).

An inhibitory nucleic acid targeting APOE gene isoform(s) (e.g., APOE4, APOE3 and/or APOE2) may comprise a region of complementarity (e.g., a region of the inhibitory nucleic acid that hybridizes to the target gene, for example a gene encoding APOE4 APOE3 and/or APOE2) that is between 6 and 50 nucleotides in length. In some embodiments, an inhibitory nucleic acid comprises a region of complementarity with APOE that is between about 6 and 30, about 8 and 20, or about 10 and 19 nucleotides in length. In some embodiments, an inhibitory nucleic acid is complementary with at least 3, 4, 5, 6, 7, 8, 9, 10, 11, 12, 13, 14, 15, 16, 17, 18, 19, 20, 21, 22, 23, 24, or 25 contiguous nucleotides of a APOE sequence. In some embodiments, an inhibitory nucleic acid targeting an APOE gene is non-allele-specific (e.g., the inhibitory nucleic acid silences all isoforms of APOE gene). In some embodiments, an inhibitory nucleic acid targets one or more specific alleles of APOE, for example one or more of APOE2, APOE3, and/or APOE4. In some embodiments, an inhibitory nucleic acid does not target (e.g., does not inhibit expression or activity of) an APOE2ch or APOE3ch isoform.

In some embodiments, a gene product (e.g., an inhibitory RNA) hybridizes to portion of a target gene (e.g., is complementary to 5, 6, 7, 8, 9, 10, 11, 12, 13, 14, 15, 16, 17, 18, 19, 20, 21, or more contiguous nucleotides of a target gene, for example APOE4 isoform of APOE, such as the sequence set forth in SEQ ID NO: 1). In some embodiments, a gene product (e.g., an inhibitory RNA) hybridizes to portion of a target gene (e.g., is complementary to 5, 6, 7, 8, 9, 10, 11, 12, 13, 14, 15, 16, 17, 18, 19, 20, 21, or more contiguous nucleotides of a target gene, for example APOE2 isoform of APOE, such as the sequence set forth in SEQ ID NO: 2). In some embodiments, a gene product (e.g., an inhibitory RNA) hybridizes to portion of a target gene (e.g., is complementary to 5, 6, 7, 8, 9, 10, 11, 12, 13, 14, 15, 16, 17, 18, 19, 20, 21, or more contiguous nucleotides of a target gene, for example APOE3 isoform of APOE, such as the sequence set forth in SEQ ID NO: 4).

In some embodiments, an expression construct is monocistronic (e.g., the expression construct encodes a single fusion protein comprising a first gene product and a second gene product). In some embodiments, an expression construct is polycistronic (e.g., the expression construct encodes two distinct gene products, for example two different proteins or protein fragments).

A polycistronic expression vector may comprise a one or more (e.g., 1, 2, 3, 4, 5, or more) promoters. Any suitable promoter can be used, for example, a constitutive promoter, an inducible promoter, an endogenous promoter, a tissue-specific promoter (e.g., a CNS-specific promoter), etc. In some embodiments, a promoter is a chicken beta-actin promoter (CBA promoter), a CAG promoter (for example as described by Alexopoulou et al. (2008) BMC Cell Biol. 9:2; doi: 10.1186/1471-2121-9-2), a CD68 promoter, or a JeT promoter (for example as described by Tornse et al. (2002) Gene 297(1-2):21-32). In some embodiments, a promoter is operably-linked to a nucleic acid sequence encoding a first gene product, a second gene product, or a first gene product and a second gene product. In some embodiments, an expression cassette comprises one or more additional regulatory sequences, including but not limited to transcription factor binding sequences, intron splice sites, poly(A) addition sites, enhancer sequences, repressor binding sites, or any combination of the foregoing.

In some embodiments, a nucleic acid sequence encoding a first gene product and a nucleic acid sequence encoding a second gene product are separated by a nucleic acid sequence encoding an internal ribosomal entry site (IRES). Examples of IRES sites are described, for example, by Mokrejs et al. (2006) Nucleic Acids Res. 34(Database issue):D125-30. In some embodiments, a nucleic acid sequence encoding a first gene product and a nucleic acid sequence encoding a second gene product are separated by a nucleic acid sequence encoding a self-cleaving peptide. Examples of self-cleaving peptides include but are not limited to T2A, P2A, E2A, F2A, BmCPV 2A, and BmIFV 2A, and those described by Liu et al. (2017) Sci Rep. 7: 2193. In some embodiments, the self-cleaving peptide is a T2A peptide.

In some embodiments, disorders such as AD are associated with the expression of at least one copy of APOE4. Accordingly, in some embodiments, isolated nucleic acids described herein comprise an inhibitory nucleic acid that reduces or prevents the expression of APOE4 (e.g., APOE). A sequence encoding an inhibitory nucleic acid may be placed in an untranslated region (e.g., intron, 5′UTR, 3′UTR, etc.) of an expression vector.

In some embodiments, an inhibitory nucleic acid is positioned in an intron of an expression construct, for example in an intron upstream of the sequence encoding a first gene product. An inhibitory nucleic acid can be a double stranded RNA (dsRNA), shRNA, siRNA, micro-RNA (miRNA), artificial miRNA (amiRNA), or an RNA aptamer. Generally, an inhibitory nucleic acid binds to (e.g., hybridizes with) between about 6 and about 30 (e.g., any integer between 6 and 30, inclusive) contiguous nucleotides of a target RNA (e.g., mRNA). In some embodiments, the inhibitory nucleic acid molecule is a miRNA or an amiRNA, for example a miRNA that targets the APOE4 isoform of APOE (the gene encoding APOE4 protein). In some embodiments, the inhibitory nucleic acid molecule is a miRNA or an amiRNA, for example a miRNA that targets the APOE3 isoform of APOE (the gene encoding APOE3 protein). In some embodiments, the inhibitory nucleic acid molecule is a miRNA or an amiRNA, for example a miRNA that targets the APOE2 isoform of APOE (the gene encoding APOE2 protein). In some embodiments, the miRNA does not comprise any mismatches with the region of APOE mRNA to which it hybridizes (e.g., the miRNA is “perfected”). In some embodiments, the inhibitory nucleic acid is an shRNA (e.g., an shRNA targeting APOE), for example encoded by in any one of SEQ ID NOs: 12-23. In some embodiments, a miRNA comprises at least one (e.g., 1, 2, 3, 4, 5, 6, 7, 8, 9, 10, or more) mismatches with the region of APOE mRNA to which it hybridizes.

In some embodiments, an inhibitory nucleic acid is an artificial microRNA (amiRNA). A microRNA (miRNA) typically refers to a small, non-coding RNA found in plants and animals and functions in transcriptional and post-translational regulation of gene expression. MiRNAs are transcribed by RNA polymerase to form a hairpin-loop structure referred to as a pri-miRNAs which are subsequently processed by enzymes (e.g., Drosha, Pasha, spliceosome, etc.) to for a pre-miRNA hairpin structure which is then processed by Dicer to form a miRNA/miRNA* duplex (where * indicates the passenger strand of the miRNA duplex), one strand of which is then incorporated into an RNA-induced silencing complex (RISC). In some embodiments, an inhibitory RNA as described herein is a miRNA targeting the APOE4 isoform of APOE (the gene encoding APOE4 protein). In some embodiments, an inhibitory RNA as described herein is a miRNA targeting the APOE3 isoform of APOE (the gene encoding APOE3 protein). In some embodiments, an inhibitory RNA as described herein is a miRNA targeting the APOE2 isoform of APOE (the gene encoding APOE2 protein).

In some embodiments, an inhibitory nucleic acid targeting APOE (e.g., the APOE4 isoform, APOE3 isoform, or APOE2 isoform of APOE) comprises a miRNA/miRNA* duplex. In some embodiments, the miRNA strand of a miRNA/miRNA* duplex comprises or consists of the sequence encoded by any one of SEQ ID NOs: 12-23. In some embodiments, the miRNA* strand of a miRNA/miRNA* duplex comprises or consists of the sequence encoded by any one of SEQ ID NOs: 12-23.

An artificial microRNA (amiRNA) is derived by modifying native miRNA to replace natural targeting regions of pre-mRNA with a targeting region of interest. For example, a naturally occurring, expressed miRNA can be used as a scaffold or backbone (e.g., a pri-miRNA scaffold), with the stem sequence replaced by that of a miRNA targeting a gene of interest. An artificial precursor microRNA (pre-amiRNA) is normally processed such that one single stable small RNA is preferentially generated. In some embodiments, rAAV vectors and rAAVs described herein comprise a nucleic acid encoding an amiRNA. In some embodiments, the pri-miRNA scaffold of the amiRNA is derived from a pri-miRNA selected from the group consisting of pri-MIR-21, pri-MIR-22, pri-MIR-26a, pri-MIR-30a, pri-MIR-33, pri-MIR-122, pri-MIR-375, pri-MIR-199, pri-MIR-99, pri-MIR-194, pri-MIR-155, and pri-MIR-451. In some embodiments, an amiRNA comprises a nucleic acid sequence targeting APOE (e.g., APOE4 isoform of APOE) and an eSIBR amiRNA scaffold, for example as described in Fowler et al. Nucleic Acids Res. 2016 Mar. 18; 44(5): e48.

In some embodiments, an amiRNA targeting APOE (e.g., the APOE4 isoform, APOE3 isoform, or APOE2 isoform of APOE) comprises or consists of the sequence encoded by any one of SEQ ID NOs: 15, 19, and 23.

An isolated nucleic acid as described herein may exist on its own, or as part of a vector. Generally, a vector can be a plasmid, cosmid, phagemid, bacterial artificial chromosome (BAC), or a viral vector (e.g., adenoviral vector, adeno-associated virus (AAV) vector, retroviral vector, baculoviral vector, etc.). In some embodiments, the vector is a plasmid (e.g., a plasmid comprising an isolated nucleic acid as described herein). In some embodiments, the vector is a recombinant AAV (rAAV) vector. An rAAV may comprise either the “plus strand” or the “minus strand” of an rAAV vector. In some embodiments, an rAAV vector is single-stranded (e.g., single-stranded DNA). In some embodiments, a vector is a Baculovirus vector (e.g., an Autographa californica nuclear polyhedrosis (AcNPV) vector).

Typically, an rAAV vector comprises a transgene (e.g., an expression construct comprising one or more of each of the following: promoter, intron, enhancer sequence, protein coding sequence, inhibitory RNA coding sequence, polyA tail sequence, etc.) flanked by two AAV inverted terminal repeat (ITR) sequences. In some embodiments the transgene of an rAAV vector comprises an isolated nucleic acid as described by the disclosure. In some embodiments, each of the two ITR sequences of an rAAV vector is a full-length ITR (e.g., approximately 145 bp in length, and containing functional Rep binding site (RBS) and terminal resolution site (trs)). In some embodiments, one of the ITRs of an rAAV vector is truncated (e.g., shortened or not full-length). In some embodiments, a truncated ITR lacks a functional terminal resolution site (trs) and is used for production of self-complementary AAV vectors (scAAV vectors). In some embodiments, a truncated ITR is a AITR, for example as described by McCarty et al. (2003) Gene Ther. 10(26):2112-8.

Aspects of the disclosure relate to isolated nucleic acids (e.g., rAAV vectors) comprising an ITR having one or more modifications (e.g., nucleic acid additions, deletions, substitutions, etc.) relative to a wild-type AAV ITR, for example relative to wild-type AAV2 ITR (e.g., SEQ ID NO: 24). Generally, a wild-type ITR comprises a 125-nucleotide region that self-anneals to form a palindromic double-stranded T-shaped, hairpin structure consisting of two cross arms (formed by sequences referred to as B/B′ and C/C′, respectively), a longer stem region (formed by sequences A/A′), and a single-stranded terminal region referred to as the “D” region. Generally, the “D” region of an ITR is positioned between the stem region formed by the A/A′ sequences and the insert containing the transgene of the rAAV vector (e.g., positioned on the “inside” of the ITR relative to the terminus of the ITR or proximal to the transgene insert or expression construct of the rAAV vector). The “D” region has been observed to play an important role in encapsidation of rAAV vectors by capsid proteins, for example as disclosed by Ling et al. (2015) J Mol Genet Med 9(3).

An isolated nucleic acid or rAAV vector as described by the disclosure may further comprise a “TRY” sequence, for example as described by Francois, et al. 2005. J Virol, The Cellular TATA Binding Protein Is Required for Rep-Dependent Replication of a Minimal Adeno-Associated Virus Type 2 p5 Element. In some embodiments, a TRY sequence is positioned between an ITR (e.g., a 5′ ITR) and an expression construct (e.g., a transgene-encoding insert) of an isolated nucleic acid or rAAV vector.

In some aspects, the disclosure relates to Baculovirus vectors comprising an isolated nucleic acid or rAAV vector as described by the disclosure. In some embodiments, the Baculovirus vector is an Autographa californica nuclear polyhedrosis (AcNPV) vector, for example as described by Urabe et al. (2002) Hum Gene Ther 13(16):1935-43 and Smith et al. (2009) Mol Ther 17(11):1888-1896.

In some aspects, the disclosure provides a host cell comprising an isolated nucleic acid or vector as described herein. A host cell can be a prokaryotic cell or a eukaryotic cell. For example, a host cell can be a mammalian cell, bacterial cell, yeast cell, insect cell, etc. In some embodiments, a host cell is a mammalian cell, for example a HEK293T cell. In some embodiments, a host cell is a bacterial cell, for example an E. coli cell.

rAAVs

In some aspects, the disclosure relates to recombinant AAVs (rAAVs) comprising a transgene that encodes a nucleic acid as described herein (e.g., an rAAV vector as described herein). The term “rAAVs” generally refers to viral particles comprising an rAAV vector encapsidated by one or more AAV capsid proteins. An rAAV described by the disclosure may comprise a capsid protein having a serotype selected from AAV1, AAV2, AAV3, AAV4, AAV5, AAV6, AAV7, AAV8, AAV9, and AAV10. In some embodiments, an rAAV comprises a capsid protein from a non-human host, for example a rhesus AAV capsid protein such as AAVrh.10, AAVrh.39, etc. In some embodiments, an rAAV described by the disclosure comprises a capsid protein that is a variant of a wild-type capsid protein, such as a capsid protein variant that includes at least 1, 2, 3, 4, 5, 6, 7, 8, 9, 10, or more than 10 (e.g., 15, 20, 25, 50, 100, etc.) amino acid substitutions (e.g., mutations) relative to the wild-type AAV capsid protein from which it is derived.

In some embodiments, rAAVs described by the disclosure readily spread through the CNS, particularly when introduced into the CSF space or directly into the brain parenchyma. Accordingly, in some embodiments, rAAVs described by the disclosure comprise a capsid protein that is capable of crossing the blood-brain barrier (BBB). For example, in some embodiments, an rAAV comprises a capsid protein having an AAV9 or AAVrh.10 serotype. Production of rAAVs is described, for example, by Samulski et al. (1989) J Virol. 63(9):3822-8 and Wright (2009) Hum Gene Ther. 20(7): 698-706.

In some embodiments, an rAAV as described by the disclosure (e.g., comprising a recombinant rAAV genome encapsidated by AAV capsid proteins to form an rAAV capsid particle) is produced in a Baculovirus vector expression system (BEVS). Production of rAAVs using BEVS are described, for example by Urabe et al. (2002) Hum Gene Ther 13(16):1935-43, Smith et al. (2009) Mol Ther 17(11):1888-1896, U.S. Pat. Nos. 8,945,918, 9,879,282, and International PCT Publication WO 2017/184879. However, an rAAV can be produced using any suitable method (e.g., using recombinant rep and cap genes).

### Pharmaceutical Compositions

In some aspects, the disclosure provides pharmaceutical compositions comprising an isolated nucleic acid or rAAV as described herein and a pharmaceutically acceptable carrier. As used herein, the term “pharmaceutically acceptable” refers to a material, such as a carrier or diluent, which does not abrogate the biological activity or properties of the compound, and is relatively non-toxic, e.g., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

Compositions (e.g., pharmaceutical compositions) provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

### Methods

The disclosure is based, in part, on compositions for expression of combinations of AD-associated gene products in a subject that act together (e.g., synergistically) to treat Alzheimer's disease. As used herein “treat” or “treating” refers to (a) preventing or delaying onset of Alzheimer's disease; (b) reducing severity of Alzheimer's disease; (c) reducing or preventing development of symptoms characteristic of Alzheimer's disease; (d) and/or preventing worsening of symptoms characteristic of Alzheimer's disease. Symptoms of Alzheimer's disease include, for example, cognitive dysfunction (e.g., dementia, hallucination, memory loss, etc.), motor dysfunction (e.g., difficulty performing daily tasks, etc.), and emotional and behavioral dysfunction.

Accordingly, in some aspects, the disclosure provides a method comprising administering to a subject having or suspected of having Alzheimer's disease (e.g., ADAD) a composition (e.g., a composition comprising an isolated nucleic acid or a vector or a rAAV) as described herein. As used herein, the terms “administering” or “administration” means to provide a composition (e.g., a composition comprising an isolated nucleic acid or a vector or a rAAV) to a subject in a manner that is physiologically and/or pharmacologically useful (e.g., to treat a condition such as AD in the subject). In some aspects, the disclosure provides a method for treating a subject having or suspected of having Alzheimer's disease (e.g., ADAD), the method comprising administering to the subject a composition (e.g., a composition comprising an isolated nucleic acid or a vector or a rAAV) as described by the disclosure.

In some embodiments, the administration of a composition (e.g., a composition comprising an isolated nucleic acid or a vector or a rAAV) as described herein results in delayed onset of mild cognitive impairment (MIC). Mild cognitive impairment (MIC) is an early stage of memory loss or other cognitive ability loss (such as language or visual/spatial perception) in subjects (e.g., AD patients) who maintain the ability to independently perform most activities of daily living. In some embodiments, the administration of a composition (e.g., a composition comprising an isolated nucleic acid or a vector or a rAAV) as described herein results in delayed onset of mild cognitive impairment (MIC) by more than one month, more than two months, more than three months, more than four months, more than five months, more than six months, more than seven months, more than eight months, more than nine months, more than ten months, more than eleven months, more than twelve months, more than one year, more than two years, more than three years, more than four years, more than five years, more than six years, more than seven years, more than eight years, more than nine years, or more than ten years as compared to subjects not administered with the composition described herein. In some embodiments, the administration of a composition (e.g., a composition comprising an isolated nucleic acid or a vector or a rAAV) as described herein results in delayed onset of mild cognitive impairment (MIC) by between one and three months, between one and six months, between three and six months, between three and nine months, between six and nice months, between one and twelve months, between six and twelve months, between one and two years, between one and three years, between one and four years, between one and five years, between one and six years, between one and seven years, between one and eight years, between one and nine years, between one and ten years, between ten and twenty years, or more as compared to subjects not administered with the composition described herein.

A subject is typically a mammal, for example a human, dog, cat, pig, hamster, rat, mouse, etc. In some embodiments, a subject is a human. In some embodiments, a subject is characterized by a Presenilin 1 (PSEN1) E280A mutation allele. A subject may be homozygous (e.g., PSEN1 E280A^{+/+}) or heterozygous (e.g., PSEN1 E280A^{+/+}) for PSEN1 E280A mutation allele. In some embodiments, a subject having a Presenilin 1 (PSEN1) E280A mutation is not a homozygote for APOE3 Christchurch mutation (e.g., APOE3 R136S^{+/-} or APOE3 R136S^{-/-}).

In some embodiments, a subject is characterized by an APOE4 allele. A subject may be homozygous (e.g., APOE4^{+/+}) or heterozygous for APOE4 (e.g., APOE4^{+/-}). In some embodiments, a subject is heterozygous for APOE4 and the second APOE allele of the subject is selected from APOE2 and APOE3.

In some embodiments, a composition is administered directly to the CNS of the subject, for example by direct injection into the brain and/or spinal cord of the subject. Examples of CNS-direct administration modalities include but are not limited to intracerebral injection, intraventricular injection, intracisternal injection, intraparenchymal injection, intrathecal injection, and any combination of the foregoing. In some embodiments, direct injection into the CNS of a subject results in transgene expression (e.g., expression of the first gene product, second gene product, and if applicable, third gene product) in the midbrain, striatum and/or cerebral cortex of the subject. In some embodiments, direct injection into the CNS results in transgene expression (e.g., expression of the first gene product, second gene product, and if applicable, third gene product) in the spinal cord and/or CSF of the subject.

In some embodiments, direct injection to the CNS of a subject comprises convection enhanced delivery (CED). Convection enhanced delivery is a therapeutic strategy that involves surgical exposure of the brain and placement of a small-diameter catheter directly into a target area of the brain, followed by infusion of a therapeutic agent (e.g., a composition or rAAV as described herein) directly to the brain of the subject. CED is described, for example by Debinski et al. (2009) Expert Rev Neurother. 9(10):1519-27.

In some embodiments, a composition is administered peripherally to a subject, for example by peripheral injection. Examples of peripheral injection include subcutaneous injection, intravenous injection, intra-arterial injection, intraperitoneal injection, or any combination of the foregoing. In some embodiments, the peripheral injection is intra-arterial injection, for example injection into the carotid artery of a subject.

In some embodiments, a composition (e.g., a composition comprising an isolated nucleic acid or a vector or a rAAV) as described by the disclosure is administered both peripherally and directly to the CNS of a subject. For example, in some embodiments, a subject is administered a composition by intra-arterial injection (e.g., injection into the carotid artery) and by intraparenchymal injection (e.g., intraparenchymal injection by CED). In some embodiments, the direct injection to the CNS and the peripheral injection are simultaneous (e.g., happen at the same time). In some embodiments, the direct injection occurs prior (e.g., between 1 minute and 1 week, or more before) to the peripheral injection. In some embodiments, the direct injection occurs after (e.g., between 1 minute and 1 week, or more after) the peripheral injection.

The amount of composition (e.g., a composition comprising an isolated nucleic acid or a vector or a rAAV) as described by the disclosure administered to a subject will vary depending on the administration method. For example, in some embodiments, a rAAV as described herein is administered to a subject at a titer between about 10⁹ Genome copies (GC)/kg and about 10¹⁴ GC/kg (e.g., about 10⁹ GC/kg, about 10¹⁰ GC/kg, about 10¹¹ GC/kg, about 10¹² GC/kg, about 10¹² GC/kg, or about 10¹⁴ GC/kg). In some embodiments, a subject is administered a high titer (e.g., >10¹² Genome Copies GC/kg of an rAAV) by injection to the CSF space, or by intraparenchymal injection.

A composition (e.g., a composition comprising an isolated nucleic acid or a vector or a rAAV) as described by the disclosure can be administered to a subject once or multiple times (e.g., 2, 3, 4, 5, 6, 7, 8, 9, 10, 20, or more) times. In some embodiments, a composition is administered to a subject continuously (e.g., chronically), for example via an infusion pump.

In some embodiments, a composition (e.g., a composition comprising an isolated nucleic acid or a vector or a rAAV) described herein can be administered to a subject in combination with another suitable therapeutic agents (e.g., therapeutic agents for treating AD). Non-limiting examples of other suitable therapeutic agents for treating AD include amyloid-β antibodies (e.g., aducanumab, bapineuzumab and solanezumab), Donepezil, Galantamine, Rivastigmine, Memantine, Suvorexant etc.

### EXAMPLES

### Example 1: Protective Role of APOE3 Christchurch Homozygotes in Autosomal Dominant Alzheimer's Disease (ADAD)

Presenilin 1 (PSEN1) E280A mutation causes the autosomal dominant Alzheimer's disease (ADAD). Although there is some variability in the age at clinical onset and disease course, patients that are carriers for the PSEN1 E280A mutation develop mild cognitive impairment (MCI) and dementia at the respective median ages of 44 (95% CI, 43-45) and 49 (95% CI, 49-50) years. It was discovered that a subject having the PSEN1 E280A mutation did not develop MCI until her seventies, nearly three decades after the typical age of onset. The subject's memory deficits were limited to recent events and her neurological examinations were normal. Whole-exome sequencing corroborated her PSEN1 E280A mutation and revealed that she had two copies of the rare Christchurch (APOEch) mutation (an arginine-to-serine substitution at amino acid 136, corresponding to codon 154) in APOE3. It was confirmed that the PSEN1 E280A mutation as the primary risk factor, and APOE3ch homozygosity as the most likely genetic modifier for this subject. In a subsequent study, subjects had one copy of the APOE3ch mutation with PSEN1 E280A mutation were not protected from developing MCI at a mean age of 45. Suggesting that APOE3ch homozygosity is required to postpone the clinical onset of ADAD (see, e.g., Arboleda-Velasquez et al., Resistance to autosomal dominant Alzheimer's disease in an APOE3 Christchurch homozygote: a case report, NATURE MEDICINE, VOL 25, NOVEMBER 2019, p. 1680-1683).

APOE, the major susceptibility gene for late-onset Alzheimer's disease, has three common alleles (APOE2, APOE3, and APOE4). APOE3 was previously thought to be neutral with regard to Alzheimer's disease risk. APOE2 is associated with a lower risk of Alzheimer's disease and older age at onset of dementia, and each additional copy of APOE4 is associated with a higher risk and younger age at onset.

Interestingly, the subject having APOE3ch homozygotes showed much higher amyloid-β plaque burden than that in PSEN1 E280A carriers who are not APOE3ch homozygotes. Despite the high amyloid-β plaque burden, the magnitude and spatial extent of her PHF tau burden and neurodegeneration were relatively limited. The tau burden of this subject was limited to medial temporal and occipital regions, with relative sparing of other regions that are characteristically affected in the clinical stages of Alzheimer's disease. Further, the cerebral metabolic rate for glucose was preserved in this subject in regions that are known to be preferentially affected by Alzheimer's disease. Magnetic resonance imaging showed that this subject had the same extent of brain atrophy as compared to other PSEN1 E280A carriers who developed MCI in their forties. The subject also had low plasma neurofilament light chain (NfL)—a marker for familial Alzheimer's disease. These observations suggested that APOE3ch homozygote elicit the protective role by limiting tau pathology and neurodegeneration despite the high amyloid-β plaque burden.

It was later confirmed that Aβ42 aggregation was reduced in the presence of APOE3ch protein as compared to Aβ42 aggregation in wild-type APOE3 protein. Aβ42 aggregation level was similar in the presence of APOE3ch and APOE2. These results suggest that APOE3ch is less capable of triggering Aβ42 aggregation.

The R136S mutation is located in a region of APOE known to have a role in binding to lipoprotein receptors (LDLR) and heparan sulfate proteoglycans (HSPGs). Previous reports showed that compared to APOE3, APOE2 and APOE3ch are associated with 98% and 60% reduction in LDLR binding, respectively. It has been suggested that APOE binding to HSPGs are necessary for HSPGs to promote amyloid-β aggregation and neuronal uptake of extracellular tau. It was observed that compared to other APOE isoforms, APOE3ch displayed the lowest heparin binding ability, and antibody blocking of APOE-HSPG interaction reproduces the protective effect of APOE3ch.

The present disclosure, at least in part, is based on the discovery of the protective role of APOE3ch in subjects having the PSEN1 E280A mutation. Gene therapy delivery of APOE3ch to PSEN1 E280A carriers (e.g., PSEN1 E280A carriers who are not APOE3ch homozygotes) may confer benefits such as delaying MIC onset, reducing tau pathology, etc.

This example describes isolated nucleic acids (e.g. vectors, such as rAAV vectors and rAAVs containing isolated nucleic acids) comprising an expression construct encoding an APOE Christchurch protein to overexpress the APOE Christchurch protein. The APOE Christchurch protein can be a recombinant APOE2 Christchurch protein (APOE2ch) or a recombinant APOE3 Christchurch protein (APOE3ch). The APOE2ch and/or the APOE3ch coding sequences codon-optimized to differ sufficiently from the endogenous APOE2 sequence in cells such that it would not be recognized by shRNAs targeting wild-type APOE, regardless of isoform.

The isolated nucleic acid can further include coding sequences for inhibitory nucleic acid targeting one or more APOE gene isoforms (e.g., APOE4, and/or APOE3, and/or APOE2). In some embodiments, constructs described in this example are useful for treating a subject having or suspected of having Alzheimer's disease (AD) (e.g., autosomal dominant Alzheimer's disease) who are carriers of the PSEN1 E280A mutation. In some embodiments, a subject is not homozygous for APOE3 Christchurch mutation (e.g., APOE3 R136S^{+/+}).

Isolated nucleic acids encoding shRNAs are utilized to knockdown the expression of the APOE4 and/or APOE2 isoform specifically both in vitro and in vivo. In some embodiments, the shRNAs are non-allele-specific, e.g., they are also capable of knocking down expression of other APOE isoforms (e.g., E2, E3, or E4).

The shRNA and transgene coding sequences can be operably linked to the same or to separate promoters. shRNAs are expressed under a separate promoter, typically a Pol III promoter (e.g., H1 promoter), or a Pol II promoter (e.g., CBA, T7, etc.). Generally, the shRNA is operably-linked to a Pol II promoter placed in an intronic sequence upstream of an open reading frame comprising the codon-optimized APOE2ch and/or APOE3ch transgene.

Recombinant adeno-associated viruses (rAAVs) comprising the isolated nucleic acids are generated using cells, such as HEK293 cells for triple-plasmid transfection. The ITR sequences flank an expression construct, which typically comprises one or more of the following: at least one promoter/enhancer element, a 3′ polyA signal, and posttranslational signals such as the WPRE element. Multiple gene products are expressed simultaneously such as the APOE2ch and/or the APOE3ch protein and one or more inhibitory nucleic acids (e.g., inhibitory nucleic acids targeting the APOE4 and/or APOE2 isoforms of APOE). The presence of a short intronic sequence that is efficiently spliced, upstream of the expressed gene, can improve expression levels. shRNAs and other regulatory RNAs can potentially be included within these sequences.

### Example 2: Cell Based Assays of Viral Transduction into APOE4^{+/+} Cells

Cells are obtained, for example as fibroblasts from ADAD patients, monocytes, or hES cells, or patient-derived induced pluripotent stem cells (iPSCs). These cells accumulate proteinaceous plaques comprising amyloid-β protein and tangles comprising twisted strands of the protein Tau.

Using such cell models, neurodegenerative characteristics associated with ADAD are quantified in terms of accumulation of protein aggregates such as plaques and tangles, for example, utilizing an α-amyloid-β antibody or α-phospho-Tau antibody, followed by imaging using fluorescent microscopy. Imaging for neurodegenerative characteristics associated with ADAD by ICC for protein markers such as amyloid-β, phospho-Tau, PSEN1 E280A, APOE3, APOE3ch, or APOE4 is also performed. Western blotting, ELISA, and/or qPCR is used to quantify APOE3ch expression levels in these cells.

Therapeutic endpoints (e.g., reduction of ADAD-associated pathology) are measured in the context of expression of transduction of the rAAVs, to confirm and quantify activity and function. The levels of amyloid-β and phospho-Tau are also quantified using Western blotting, ELISA, and/or qPCR.

### Example 3: Clinical Trials in ADAD Patients

This example describes clinical trials to assess the safety and efficacy of rAAVs as described by the disclosure, in patients having ADAD (e.g., PSEN1 E280A carriers who are not APOE3ch homozygotes).

Clinical trials of rAAVs of the present disclosure for treatment of ADAD are performed using a study design similar to that described in Grabowski et al. (1995) Ann. Intern. Med. 122(1):33-39. The rAAVs are delivered into the CSF, intraparenchymally to the hippocampus or to another brain region, or peripherally.

Endpoints measured are levels of amyloid-β plaques, Tau tangles, motor and cognitive endpoints, and levels of APOE3ch, APOE4, and APOE2 proteins.

### Example 4: Clinical Trials in ADAD Patients Combined with Amyloid-β Antibodies

This example describes clinical trials to assess the safety and efficacy of rAAVs as described by the disclosure, utilized in combination with amyloid-β antibodies (e.g., bapineuzumab and solanezumab) in patients having ADAD (e.g., PSEN1 E280A carriers who are not APOE3ch homozygotes).

Clinical trials of rAAVs of the present disclosure, combined with anti-amyloid-β antibodies, for treatment of ADAD are performed using a study design similar to that described in Grabowski et al. (1995) Ann. Intern. Med. 122(1):33-39. The rAAVs are delivered into the CSF, intraparenchymally to the hippocampus or to another brain region, or peripherally.

In some embodiments, rAAVs of the disclosure synergize with anti-amyloid-β antibodies to reduce the likelihood of ADAD patients developing amyloid-related imaging abnormalities (ARIA), which are highly correlated with APOE genotype. ARIAs are a spectrum of abnormalities observed in AD patients which are associated with amyloid-modifying therapies, particularly with human monoclonal antibodies. There are two types of ARIAs, ARIA-E, which refers to cerebral edema, and ARIA-H, which refers to cerebral microhemmorhages.

Endpoints evaluated are brain imaging before and after treatment to determine if ARIA has occurred and whether rAAVs of the disclosure reduce the likelihood of ARIA, levels of amyloid-β plaques, Tau tangles, motor and cognitive endpoints, and levels of APOE3ch, APOE4 and APOE2 proteins.

### Example 5: Clinical Trials in ADAD Patients Having the PSEN1 E280A Mutation Who are APOE3ch^{+/+}, APOE3ch^{+/-}, and APO3ch^{-/-}

This example describes clinical trials to assess the efficacy of rAAVs as described by the disclosure in ameliorating the increased risk of other pathologies including stroke, coronary artery disease, atherosclerosis, poor recovery from head trauma, and cognitive recovery from surgery on a bypass machine, in patients having the PSEN1 E280A mutation who are not APOE3ch^{+/+}, compared to patients who are APOE3ch^{+/-}, or APO3ch^{-/-}.

Clinical trials of rAAVs of the disclosure for treatment of AD and ameliorating increased risk of other conditions associated with patients having the PSEN1 E280A mutation who are APOE3ch^{+/-}, or APO3ch^{-/-} are performed using a study design similar to that described in Grabowski et al. (1995) Ann. Intern. Med. 122(1):33-39. The rAAVs are delivered into the CSF, intraparenchymally to the hippocampus or to another brain region, or peripherally.

Endpoints evaluated before and after treatment with rAAVs of the disclosure are blood pressure, blood cholesterol and blood sugar levels, motor and cognitive endpoints, MRI, PET, and ultrasound imaging of the coronary arteries, recovery from cognitive trauma, and recovery from surgery on a bypass machine.

### Example 6: Prevention of ADAD or Treatment of ADAD in Patient Carriers of the PSEN1 E280A Mutation

This example describes clinical trials to assess the efficacy of rAAVs as described by the disclosure in reducing the risk of subjects having the PSEN1 E280A mutation developing AD and in treating AD in patients with the PSEN1 E280A mutation. Patients with the PSEN1 E280A mutation can be either APOE3ch^{+/-} or APOE3ch^{-/-}.

Clinical trials of rAAVs of the present disclosure for the prevention or treatment of AD in carriers of the PSEN1 E280A mutation are performed using a study design similar to that described in Grabowski et al. (1995) Ann. Intern. Med. 122(1):33-39. The rAAVs are delivered into the CSF, intraparenchymally to the hippocampus or to another brain region, or peripherally.

Endpoints evaluated before and after treatment with rAAVs of the present disclosure are the levels of APOE3ch, APOE4 and APOE2 in the CSF and the blood and cognitive and motor endpoints.

### Example 7: In Vitro Validation of shRNAs for Endogenous APOE Silencing and APOE Christchurch Protein Over Expression

Multiple plasmids containing unique shRNAs against APOE and codon-optimized coding sequence of APOE Christchurch protein (e.g., APOE3ch and/or APOE2ch) are evaluated in in vitro transfection screens to assess the extent of APOE (e.g., APOE4 and/or APOE2) knockdown and heterologous expression of APOE Christchurch protein (e.g., APOE3ch and/or APOE2ch). Plasmids are specifically designed to selectively knock down the endogenous APOE gene without affecting vector-encoded APOE Christchurch protein (e.g., APOE3ch and/or APOE2ch). Multiple plasmids show reduction of endogenous APOE and expression of APOE Christchurch protein (e.g., APOE3ch and/or APOE2ch) via qRT-PCR. The shRNA candidates show significant reduction of endogenous APOE without affecting the expression of APOE Christchurch protein (e.g., APOE3ch and/or APOE2ch).

### Example 8: In Vivo Validation of shRNAs for Endogenous APOE Silencing and APOE Christchurch Protein (e.g., APOE3ch and/or APOE2ch) Over Expression

The shRNA candidates demonstrating significant reduction of endogenous APOE without affecting the codon optimized APOE Christchurch protein coding sequence (e.g., APOE3ch and/or APOE2ch coding sequence) are selected for further in vivo study. APOE4 knock-in (KI) mouse model is used to evaluate the in vivo efficacy of the candidate shRNAs against APOE4. In the APOE4 KI mice, both mouse Apoe alleles are replaced by human APOE-ε4. The mice (n=5) receive vectors carrying the candidate shRNAs against APOE4 via intracerebroventricular injection (ICV) and the biodistribution of human APOE4 mRNA is analyzed 60 days post injection.

### EQUIVALENTS

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

It should also be understood that, unless clearly indicated to the contrary, in any methods claimed herein that include more than one step or act, the order of the steps or acts of the method is not necessarily limited to the order in which the steps or acts of the method are recited.

### SEQUENCES

In some embodiments, an expression cassette encoding one or more gene products (e.g., a first, second and/or third gene product) comprises or consists of a sequence set forth in any one of SEQ ID NOs: 1-24. In some embodiments, a gene product is encoded by a portion (e.g., fragment) of a sequence set forth in any one of SEQ ID NOs: 1-24. The skilled artisan recognizes that nucleic acid sequences encoding inhibitory nucleic acids may describe a sequence where all “T” have been replaced with “U” or vice versa.

> human APOE4 nucleic acid sequence
(SEQ ID NO: 1)
ATGAAGGTTCTGTGGGCTGCGTTGCTGGTCACATTCCTGG
CAGGATGCCAGGCCAAGGTGGAGCAAGCGGTGGAGACAGA
GCCGGAGCCCGAGCTGCGCCAGCAGACCGAGTGGCAGAGC
GGCCAGCGCTGGGAACTGGCACTGGGTCGCTTTTGGGATT
ACCTGCGCTGGGTGCAGACACTGTCTGAGCAGGTGCAGGA
GGAGCTGCTCAGCTCCCAGGTCACCCAGGAACTGAGGGCG
CTGATGGACGAGACCATGAAGGAGTTGAAGGCCTACAAAT
CGGAACTGGAGGAACAACTGACCCCGGTGGCGGAGGAGAC
GCGGGCACGGCTGTCCAAGGAGCTGCAGGCGGCGCAGGCC
CGGCTGGGCGCGGACATGGAGGACGTGCGCGGCCGCCTGG
TGCAGTACCGCGGCGAGGTGCAGGCCATGCTCGGCCAGAG
CACCGAGGAGCTGCGGGTGCGCCTCGCCTCCCACCTGCGC
AAGCTGCGTAAGCGGCTCCTCCGCGATGCCGATGACCTGC
AGAAGCGCCTGGCAGTGTACCAGGCCGGGGCCCGCGAGGG
CGCCGAGCGCGGCCTCAGCGCCATCCGCGAGCGCCTGGGG
CCCCTGGTGGAACAGGGCCGCGTGCGGGCCGCCACTGTGG
GCTCCCTGGCCGGCCAGCCGCTACAGGAGCGGGCCCAGGC
CTGGGGCGAGCGGCTGCGCGCGCGGATGGAGGAGATGGGC
AGCCGGACCCGCGACCGCCTGGACGAGGTGAAGGAGCAGG
TGGCGGAGGTGCGCGCCAAGCTGGAGGAGCAGGCCCAGCA
GATACGCCTGCAGGCCGAGGCCTTCCAGGCCCGCCTCAAG
AGCTGGTTCGAGCCCCTGGTGGAAGACATGCAGCGCCAGT
GGGCCGGGCTGGTGGAGAAGGTGCAGGCTGCCGTGGGCAC
CAGCGCCGCCCCTGTGCCCAGCGACAATCACTGA
> human APOE2 nucleic acid sequence
(SEQ ID NO: 2)
ATGAAGGTTCTGTGGGCTGCGTTGCTGGTCACATTCCTGG
CAGGATGCCAGGCCAAGGTGGAGCAAGCGGTGGAGACAGA
GCCGGAGCCCGAGCTGCGCCAGCAGACCGAGTGGCAGAGC
GGCCAGCGCTGGGAACTGGCACTGGGTCGCTTTTGGGATT
ACCTGCGCTGGGTGCAGACACTGTCTGAGCAGGTGCAGGA
GGAGCTGCTCAGCTCCCAGGTCACCCAGGAACTGAGGGCG
CTGATGGACGAGACCATGAAGGAGTTGAAGGCCTACAAAT
CGGAACTGGAGGAACAACTGACCCCGGTGGCGGAGGAGAC
GCGGGCACGGCTGTCCAAGGAGCTGCAGGCGGCGCAGGCC
CGGCTGGGCGCGGACATGGAGGACGTGTGCGGCCGCCTGG
TGCAGTACCGCGGCGAGGTGCAGGCCATGCTCGGCCAGAG
CACCGAGGAGCTGCGGGTGCGCCTCGCCTCCCACCTGCGC
AAGCTGCGTAAGCGGCTCCTCCGCGATGCCGATGACCTGC
AGAAGTGCCTGGCAGTGTACCAGGCCGGGGCCCGCGAGGG
CGCCGAGCGCGGCCTCAGCGCCATCCGCGAGCGCCTGGGG
CCCCTGGTGGAACAGGGCCGCGTGCGGGCCGCCACTGTGG
GCTCCCTGGCCGGCCAGCCGCTACAGGAGCGGGCCCAGGC
CTGGGGCGAGCGGCTGCGCGCGCGGATGGAGGAGATGGGC
AGCCGGACCCGCGACCGCCTGGACGAGGTGAAGGAGCAGG
TGGCGGAGGTGCGCGCCAAGCTGGAGGAGCAGGCCCAGCA
GATACGCCTGCAGGCCGAGGCCTTCCAGGCCCGCCTCAAG
AGCTGGTTCGAGCCCCTGGTGGAAGACATGCAGCGCCAGT
GGGCCGGGCTGGTGGAGAAGGTGCAGGCTGCCGTGGGCAC
CAGCGCCGCCCCTGTGCCCAGCGACAATCACTGA
> Human ApoE2 amino acid sequence
(SEQ ID NO: 3)
MKVLWAALLVTFLAGCQAKVEQAVETEPEPELRQQTEWQS
GQRWELALGRFWDYLRWVQTLSEQVQEELLSSQVTQELRA
LMDETMKELKAYKSELEEQLTPVAEETRARLSKELQAAQA
RLGADMEDVCGRLVQYRGEVQAMLGQSTEELRVRLASHLR
KLRKRLLRDADDLQKCLAVYQAGAREGAERGLSAIRERLG
PLVEQGRVRAATVGSLAGQPLQERAQAWGERLRARMEEMG
SRTRDRLDEVKEQVAEVRAKLEEQAQQIRLQAEAFQARLK
SWFEPLVEDMQRQWAGLVEKVQAAVGTSAAPVPSDNH
> Human APOE3 nucleic acid sequence
(SEQ ID NO: 4)
AGAGACGACCCGACCCGCTAGAAGACTGGCCAATCACAGG
CAGGAAGATGAAGGTTCTGTGGGCTGCGTTGCTGGTCACA
TTCCTGGCAGGATGCCAGGCCAAGGTGGAGCAAGCGGTGG
AGACAGAGCCGGAGCCCGAGCTGCGCCAGCAGACCGAGTG
GCAGAGCGGCCAGCGCTGGGAACTGGCACTGGGTCGCTTT
TGGGATTACCTGCGCTGGGTGCAGACACTGTCTGAGCAGG
TGCAGGAGGAGCTGCTCAGCTCCCAGGTCACCCAGGAACT
GAGGGCGCTGATGGACGAGACCATGAAGGAGTTGAAGGCC
TACAAATCGGAACTGGAGGAACAACTGACCCCGGTGGCGG
AGGAGACGCGGGCACGGCTGTCCAAGGAGCTGCAGGCGGC
GCAGGCCCGGCTGGGCGCGGACATGGAGGACGTGTGCGGC
CGCCTGGTGCAGTACCGCGGCGAGGTGCAGGCCATGCTCG
GCCAGAGCACCGAGGAGCTGCGGGTGCGCCTCGCCTCCCA
CCTGCGCAAGCTGCGTAAGCGGCTCCTCCGCGATGCCGAT
GACCTGCAGAAGCGCCTGGCAGTGTACCAGGCCGGGGCCC
GCGAGGGCGCCGAGCGCGGCCTCAGCGCCATCCGCGAGCG
CCTGGGGCCCCTGGTGGAACAGGGCCGCGTGCGGGCCGCC
ACTGTGGGCTCCCTGGCCGGCCAGCCGCTACAGGAGCGGG
CCCAGGCCTGGGGCGAGCGGCTGCGCGCGCGGATGGAGGA
GATGGGCAGCCGGACCCGCGACCGCCTGGACGAGGTGAAG
GAGCAGGTGGCGGAGGTGCGCGCCAAGCTGGAGGAGCAGG
CCCAGCAGATACGCCTGCAGGCCGAGGCCTTCCAGGCCCG
CCTCAAGAGCTGGTTCGAGCCCCTGGTGGAAGACATGCAG
CGCCAGTGGGCCGGGCTGGTGGAGAAGGTGCAGGCTGCCG
TGGGCACCAGCGCCGCCCCTGTGCCCAGCGACAATCACTG
AACGCCGAAGCCTGCAGCCATGCGACCCCACGCCACCCCG
TGCCTCCTGCCTCCGCGCAGCCTGCAGCGGGAGACCCTGT
CCCCGCCCCAGCCGTCCTCCTGGGGTGGACCCTAGTTTAA
TAAAGATTCACCAAGTTTCACGCA
> Human ApoE3 amino acid sequence
(SEQ ID NO: 5)
MKVLWAALLVTFLAGCQAKVEQAVETEPEPELRQQTEWQS
GQRWELALGRFWDYLRWVQTLSEQVQEELLSSQVTQELRA
LMDETMKELKAYKSELEEQLTPVAEETRARLSKELQAAQA
RLGADMEDVCGRLVQYRGEVQAMLGQSTEELRVRLASHLR
KLRKRLLRDADDLQKRLAVYQAGAREGAERGLSAIRERLG
PLVEQGRVRAATVGSLAGQPLQERAQAWGERLRARMEEMG
SRTRDRLDEVKEQVAEVRAKLEEQAQQIRLQAEAFQARLK
SWFEPLVEDMQRQWAGLVEKVQAAVGTSAAPVPSDNH
> Human ApoE3 Christchurch mutant
amino acid sequence
(SEQ ID NO: 6)
MKVLWAALLVTFLAGCQAKVEQAVETEPEPELRQQTEWQS
GQRWELALGRFWDYLRWVQTLSEQVQEELLSSQVTQELRA
LMDETMKELKAYKSELEEQLTPVAEETRARLSKELQAAQA
RLGADMEDVCGRLVQYRGEVQAMLGQSTEELRVSLASHLR
KLRKRLLRDADDLQKRLAVYQAGAREGAERGLSAIRERLG
PLVEQGRVRAATVGSLAGQPLQERAQAWGERLRARMEEMG
SRTRDRLDEVKEQVAEVRAKLEEQAQQIRLQAEAFQARLK
SWFEPLVEDMQRQWAGLVEKVQAAVGTSAAPVPSDNH
>Human ApoE3 Christchurch mutant
nucleic acid sequence
(SEQ ID NO: 7)
ATGAAGGTTCTGTGGGCTGCGTTGCTGGTCACATTCCTGG
CAGGATGCCAGGCCAAGGTGGAGCAAGCGGTGGAGACAGA
GCCGGAGCCCGAGCTGCGCCAGCAGACCGAGTGGCAGAGC
GGCCAGCGCTGGGAACTGGCACTGGGTCGCTTTTGGGATT
ACCTGCGCTGGGTGCAGACACTGTCTGAGCAGGTGCAGGA
GGAGCTGCTCAGCTCCCAGGTCACCCAGGAACTGAGGGCG
CTGATGGACGAGACCATGAAGGAGTTGAAGGCCTACAAAT
CGGAACTGGAGGAACAACTGACCCCGGTGGCGGAGGAGAC
GCGGGCACGGCTGTCCAAGGAGCTGCAGGCGGCGCAGGCC
CGGCTGGGCGCGGACATGGAGGACGTGTGCGGCCGCCTGG
TGCAGTACCGCGGCGAGGTGCAGGCCATGCTCGGCCAGAG
CACCGAGGAGCTGCGGGTGAGCCTCGCCTCCCACCTGCGC
AAGCTGCGTAAGCGGCTCCTCCGCGATGCCGATGACCTGC
AGAAGCGCCTGGCAGTGTACCAGGCCGGGGCCCGCGAGGG
CGCCGAGCGCGGCCTCAGCGCCATCCGCGAGCGCCTGGGG
CCCCTGGTGGAACAGGGCCGCGTGCGGGCCGCCACTGTGG
GCTCCCTGGCCGGCCAGCCGCTACAGGAGCGGGCCCAGGC
CTGGGGCGAGCGGCTGCGCGCGCGGATGGAGGAGATGGGC
AGCCGGACCCGCGACCGCCTGGACGAGGTGAAGGAGCAGG
TGGCGGAGGTGCGCGCCAAGCTGGAGGAGCAGGCCCAGCA
GATACGCCTGCAGGCCGAGGCCTTCCAGGCCCGCCTCAAG
AGCTGGTTCGAGCCCCTGGTGGAAGACATGCAGCGCCAGT
GGGCCGGGCTGGTGGAGAAGGTGCAGGCTGCCGTGGGCAC
CAGCGCCGCCCCTGTGCCCAGCGACAATCACTGA
> Human ApoE2 Christchurch mutant
amino acid sequence
(SEQ ID NO: 8)
MKVLWAALLVTFLAGCQAKVEQAVETEPEPELRQQTEWQS
GQRWELALGRFWDYLRWVQTLSEQVQEELLSSQVTQELRA
LMDETMKELKAYKSELEEQLTPVAEETRARLSKELQAAQA
RLGADMEDVCGRLVQYRGEVQAMLGQSTEELRVSLASHLR
KLRKRLLRDADDLQKCLAVYQAGAREGAERGLSAIRERLG
PLVEQGRVRAATVGSLAGQPLQERAQAWGERLRARMEEMG
SRTRDRLDEVKEQVAEVRAKLEEQAQQIRLQAEAFQARLK
SWFEPLVEDMQRQWAGLVEKVQAAVGTSAAPVPSDNH
>Human ApoE2 Christchurch mutant
nucleic acid sequence
(SEQ ID NO: 9)
ATGAAGGTTCTGTGGGCTGCGTTGCTGGTCACATTCCTGG
CAGGATGCCAGGCCAAGGTGGAGCAAGCGGTGGAGACAGA
GCCGGAGCCCGAGCTGCGCCAGCAGACCGAGTGGCAGAGC
GGCCAGCGCTGGGAACTGGCACTGGGTCGCTTTTGGGATT
ACCTGCGCTGGGTGCAGACACTGTCTGAGCAGGTGCAGGA
GGAGCTGCTCAGCTCCCAGGTCACCCAGGAACTGAGGGCG
CTGATGGACGAGACCATGAAGGAGTTGAAGGCCTACAAAT
CGGAACTGGAGGAACAACTGACCCCGGTGGCGGAGGAGAC
GCGGGCACGGCTGTCCAAGGAGCTGCAGGCGGCGCAGGCC
CGGCTGGGCGCGGACATGGAGGACGTGTGCGGCCGCCTGG
TGCAGTACCGCGGCGAGGTGCAGGCCATGCTCGGCCAGAG
CACCGAGGAGCTGCGGGTGAGCCTCGCCTCCCACCTGCGC
AAGCTGCGTAAGCGGCTCCTCCGCGATGCCGATGACCTGC
AGAAGTGCCTGGCAGTGTACCAGGCCGGGGCCCGCGAGGG
CGCCGAGCGCGGCCTCAGCGCCATCCGCGAGCGCCTGGGG
CCCCTGGTGGAACAGGGCCGCGTGCGGGCCGCCACTGTGG
GCTCCCTGGCCGGCCAGCCGCTACAGGAGCGGGCCCAGGC
CTGGGGCGAGCGGCTGCGCGCGCGGATGGAGGAGATGGGC
AGCCGGACCCGCGACCGCCTGGACGAGGTGAAGGAGCAGG
TGGCGGAGGTGCGCGCCAAGCTGGAGGAGCAGGCCCAGCA
GATACGCCTGCAGGCCGAGGCCTTCCAGGCCCGCCTCAAG
AGCTGGTTCGAGCCCCTGGTGGAAGACATGCAGCGCCAGT
GGGCCGGGCTGGTGGAGAAGGTGCAGGCTGCCGTGGGCAC
CAGCGCCGCCCCTGTGCCCAGCGACAATCACTGA
> Human ApoE3 Christchurch mutant
codon optimized nucleic acid sequence
(SEQ ID NO: 10)
ATGAAGGTGCTGTGGGCCGCCCTGCTGGTGACCTTCCTGG
CCGGCTGCCAGGCCAAaGTcGAaCAGGCCGTcGAGACCGA
GCCCGAGCCCGAGCTGCGCCAGCAGACCGAGTGGCAGAGC
GGCCAGCGCTGGGAGCTGGCCCTGGGCCGCTTCTGGGACT
ACCTGCGCTGGGTGCAGACCCTGAGCGAGCAGGTGCAGGA
GGAGCTGCTGAGCAGCCAGGTGACCCAGGAGCTGCGCGCC
CTGATGGACGAGACCATGAAaGAaCTcAAaGCtTAtAAGA
GCGAGCTGGAGGAGCAGCTGACCCCCGTGGCCGAGGAGAC
CCGCGCCCGCCTGAGCAAGGAGCTGCAGGCCGCCCAGGCC
CGCCTGGGCGCCGACATGGAGGACGTGTGCGGCCGCCTGG
TGCAGTACCGCGGCGAGGTGCAGGCCATGCTGGGCCAGAG
CACCGAGGAGCTGCGCGTGAGCCTGGCCAGCCACCTGCGC
AAGCTGCGCAAGCGCCTGCTGCGCGACGCCGACGACCTGC
AGAAGCGCCTGGCCGTGTACCAGGCCGGCGCCCGCGAGGG
CGCCGAGCGCGGCCTGAGCGCCATCCGCGAGCGCCTGGGC
CCCCTGGTGGAGCAGGGCCGCGTGCGCGCCGCCACCGTGG
GCAGCCTGGCCGGCCAGCCCCTGCAGGAGCGCGCCCAGGC
CTGGGGCGAGCGCCTGCGCGCCCGCATGGAGGAGATGGGC
AGCCGCACCCGCGACCGCCTGGACGAGGTGAAGGAGCAGG
TGGCCGAGGTGCGCGCCAAGCTGGAGGAGCAGGCCCAGCA
GATCCGCCTGCAGGCCGAGGCCTTCCAGGCCCGCCTGAAG
AGCTGGTTCGAGCCCCTGGTGGAGGACATGCAGCGCCAGT
GGGCCGGCCTGGTGGAGAAGGTGCAGGCCGCCGTGGGCAC
CAGCGCCGCCCCCGTGCCCAGCGACAACCACTAA
>Human ApoE2 Christchurch mutant codon
optimized nucleic acid sequence
(SEQ ID NO: 11)
ATGAAGGTGCTGTGGGCCGCCCTGCTGGTGACCTTCCTGG
CCGGCTGCCAGGCCAAaGTcGAaCAGGCCGTcGAGACCGA
GCCCGAGCCCGAGCTGCGCCAGCAGACCGAGTGGCAGAGC
GGCCAGCGCTGGGAGCTGGCCCTGGGCCGCTTCTGGGACT
ACCTGCGCTGGGTGCAGACCCTGAGCGAGCAGGTGCAGGA
GGAGCTGCTGAGCAGCCAGGTGACCCAGGAGCTGCGCGCC
CTGATGGACGAGACCATGAAaGAaCTcAAaGCtTAtAAGA
GCGAGCTGGAGGAGCAGCTGACCCCCGTGGCCGAGGAGAC
CCGCGCCCGCCTGAGCAAGGAGCTGCAGGCCGCCCAGGCC
CGCCTGGGCGCCGACATGGAGGACGTGTGCGGCCGCCTGG
TGCAGTACCGCGGCGAGGTGCAGGCCATGCTGGGCCAGAG
CACCGAGGAGCTGCGCGTGAGCCTGGCCAGCCACCTGCGC
AAGCTGCGCAAGCGCCTGCTGCGCGACGCCGACGACCTGC
AGAAGTGCCTGGCCGTGTACCAGGCCGGCGCCCGCGAGGG
CGCCGAGCGCGGCCTGAGCGCCATCCGCGAGCGCCTGGGC
CCCCTGGTGGAGCAGGGCCGCGTGCGCGCCGCCACCGTGG
GCAGCCTGGCCGGCCAGCCCCTGCAGGAGCGCGCCCAGGC
CTGGGGCGAGCGCCTGCGCGCCCGCATGGAGGAGATGGGC
AGCCGCACCCGCGACCGCCTGGACGAGGTGAAGGAGCAGG
TGGCCGAGGTGCGCGCCAAGCTGGAGGAGCAGGCCCAGCA
GATCCGCCTGCAGGCCGAGGCCTTCCAGGCCCGCCTGAAG
AGCTGGTTCGAGCCCCTGGTGGAGGACATGCAGCGCCAGT
GGGCCGGCCTGGTGGAGAAGGTGCAGGCCGCCGTGGGCAC
CAGCGCCGCCCCCGTGCCCAGCGACAACCACTAA
> ApoE shRNA 1 nucleic acid sequence
(SEQ ID NO: 12)
TTGTAGGCCTTCAACTCCTTC
> ApoE shRNA 1 nucleic acid sequence
(SEQ ID NO: 13)
GAAGGAGTTGAAGGCCTACAA
>ApoE shRNA 1 with loop
(SEQ ID NO: 14)
TTGTAGGCCTTCAACTCCTTCCATCTGTGGCTTCACTGAA
GGAGTTGAAGGCCTACAA
> ApoE amiRNA 1
(SEQ ID NO: 15)
ttgtcatcctcccacggtggccatttgttccatgtgagtg
ctagtaacaggccttgtgtcctTTGTAGGCCTTCAACTCC
TTCCATCTGTGGCTTCACTGAAGGAGTTGAAGGCCTACAA
gacaacagcatacagccttcagcaagcctcca
> ApoE shRNA 2 nucleic acid sequence
(SEQ ID NO: 16)
ctccaccgcttgctccacctt
> ApoE shRNA 2 nucleic acid sequence
(SEQ ID NO: 17)
aaggtggagcaagcggtggag
>ApoE shRNA 2 with loop
(SEQ ID NO: 18)
ctccaccgcttgctccaccttAGTGAAGCCACAGATGaag
gtggagcaagcggtggag
> ApoE amiRNA 2
(SEQ ID NO: 19)
tggaggcttgctgaaggctgtatgctgttgtcctccaccg
cttgctccaccttAGTGAAGCCACAGATGaaggtggagca
agcggtggagaggacacaaggcctgttactagcactcaca
tggaacaaatggccaccgtgggaggatgacaa
> ApoE shRNA 3 nucleic acid sequence
(SEQ ID NO: 20)
tttgtaggccttcaactcc
> ApoE shRNA 3 nucleic acid sequence
(SEQ ID NO: 21)
ggagttgaaggcctacaaa
>ApoE shRNA 3 with loop
(SEQ ID NO: 22)
tttgtaggccttcaactccAGTGAAGCCACAGATGggagt
tgaaggcctacaaa
> ApoE amiRNA 3
(SEQ ID NO: 23)
tggaggcttgctgaaggctgtatgctgttgtctttgtagg
ccttcaactccAGTGAAGCCACAGATGggagttgaaggcc
tacaaaaggacacaaggcctgttactagcactcacatgga
acaaatggccaccgtgggaggatgacaa
>Wild-type AAV2 ITR nucleic acid sequence
(SEQ ID NO: 24)
AGGAACCCCTAGTGATGGAGTTGGCCACTCCCTCTCTGCG
CGCTCGCTCGCTCACTGAGGCCGGGCGACCAAAGGTCGCC
CGACGCCCGGGCTTTGCCCGGGCGGCCTCAGTGAGCGAGC
GAGCGCGCAGAGAGGGAGTGGCCAA

What is claimed is:

1. An isolated nucleic acid comprising an expression construct comprising a nucleic acid encoding an APOE Christchurch protein.

2. The isolated nucleic acid of claim 1, wherein the APOE Christchurch protein is an APOE2 Christchurch protein.

3. The isolated nucleic acid of claim 2, wherein the APOE2 Christchurch protein comprises an amino acid sequence at least 80% identical to SEQ ID NO: 8.

4. The isolated nucleic acid of claim 2 or 3, wherein the nucleic acid sequence encoding the APOE2 Christchurch protein comprises a nucleic acid sequence at least 80% identical to SEQ ID NO: 9.

5. The isolated nucleic acid of claim 1, wherein the APOE Christchurch protein is an APOE3 Christchurch protein.

6. The isolated nucleic acid of claim 5, wherein the APOE3 Christchurch protein comprises an amino acid sequence at least 80% identical to an amino acid sequence of SEQ ID NO: 6.

7. The isolated nucleic acid of claim 5 or 6, wherein the nucleic acid sequence encoding the APOE3 Christchurch protein comprises a nucleic acid sequence at least 80% identical to SEQ ID NO: 7.

8. The isolated nucleic acid of any one of claims 1-7, wherein the expression construct further comprises a nucleic acid sequence encoding an inhibitory nucleic acid that inhibits expression or activity of an APOE gene.

9. The isolated nucleic acid of any one of claims 1-8, wherein the expression construct further comprises a nucleic acid sequence encoding an inhibitory nucleic acid that inhibits expression or activity of APOE4.

10. The isolated nucleic acid of any one of claims 1-9, wherein the expression construct further comprises a nucleic acid sequence encoding an inhibitory nucleic acid that inhibits expression or activity of APOE2.

11. The isolated nucleic acid of any one of claims 1-8, wherein the expression construct further comprises a nucleic acid sequence encoding an inhibitory nucleic acid that inhibits expression or activity of APOE4 and APOE2.

12. The isolated nucleic acid of any one of claims 8-11, wherein the inhibitory nucleic acid is encoded by a sequence set forth in any one of SEQ ID NOs: 12-23.

13. The isolated nucleic acid of any one of claims 1 to 12, wherein the expression construct further comprises a first promoter operably linked to the nucleic acid sequence encoding the APOE Christchurch protein.

14. The isolated nucleic acid of claim 13, wherein the first promoter is operably linked to the nucleic acid sequence encoding an inhibitory nucleic acid that inhibits expression or activity of the APOE gene.

15. The isolated nucleic acid of claim 13, wherein the expression construct further comprises a second promoter operably linked to the nucleic acid sequence encoding an inhibitory nucleic acid that inhibits expression or activity of the APOE gene.

16. The isolated nucleic acid of any one of claims 13-15, wherein the first promoter and/or the second promoter is independently a chicken-beta actin (CBA) promoter, a CAG promoter, a CD68 promoter, or a JeT promoter.

17. The isolated nucleic acid of any one of claims 1 to 16, wherein the expression construct is flanked by adeno-associated virus (AAV) inverted terminal repeats (ITRs).

18. The isolated nucleic acid of claim 17, wherein the ITRs are AAV2 ITRs.

19. The isolated nucleic acid of any one of claims 1 to 18, wherein the isolated nucleic acid comprises the sequence set forth in any one of SEQ ID NOs: 6-11.

20. A vector comprising the isolated nucleic acid of any one of claims 1 to 19.

21. The vector of claim 20, wherein the vector is a plasmid.

22. The vector of claim 20, wherein the vector is a viral vector, optionally wherein the viral vector is a recombinant AAV (rAAV) vector or a Baculovirus vector.

23. A recombinant adeno-associated virus (rAAV) comprising:
(i) an AAV capsid protein; and
(ii) the isolated nucleic acid of any one of claims 1 to 19, or the vector of claim 22.

24. The rAAV of claim 23, wherein the AAV capsid protein is capable of crossing the blood-brain barrier, optionally wherein the capsid protein is an AAV9 capsid protein or an AAVrh.10 capsid protein.

25. The rAAV of claim 23 or claim 24, wherein the rAAV transduces neuronal cells and non-neuronal cells of the central nervous system (CNS).

26. A host cell comprising the isolated nucleic acid of any one of claims 1 to 19, the vector of any one of claims 20 to 22, or the rAAV of any one of claims 23-25.

27. A composition comprising the isolated nucleic acid of any one of claims 1 to 19, the vector of any one of claims 20-22, or the rAAV of any one of claims 23-25.

28. The composition of claim 27, wherein the composition is a pharmaceutical composition further comprises a pharmaceutically acceptable carrier.

29. A method comprising administering to a subject having or suspected of having Alzheimer's disease an isolated nucleic acid of any one of claims 1 to 19, the vector of any one of claims 20-22, the rAAV of any one of claims 23-25, or the composition of claim 27 or 28.

30. The method of claim 29, wherein the administration comprises direct injection to the CNS of the subject, optionally wherein the direct injection comprises intracerebral injection, intraparenchymal injection, intrathecal injection, or any combination thereof.

31. The method of claim 22, wherein the direct injection to the CNS of the subject comprises convection enhanced delivery (CED).

32. The method of any one of claims 29-31, wherein the administration comprises peripheral injection, optionally wherein the peripheral injection comprises intravenous injection.

33. The method of any one of claims 29-32, wherein the subject has or is suspected of having autosomal dominant Alzheimer's disease (ADAD).

34. The method of any one of claims 29-33, wherein the subject has at least one mutation in the PSEN1 gene.

35. The method of claim 34, wherein the mutation in the PSEN1 gene causes an E280A mutation in presenilin 1 protein.

36. The method of claim 34, wherein the subject is a PSEN1 E280A mutation homozygous.

37. The method of any one of claims 29-36, wherein the subject is not a homozygote for APOE3 Christchurch mutation, wherein the APOE3 Christchurch mutation causes a R136S mutation in APOE3 protein.

38. The method of any one of claims 29-37, wherein the administration results in delayed onset of mild cognitive impairment (MIC) compared to subjects not receiving the administration.
